(19) United States
(12) Patent Application Publication
(10) Pub. No.: US 2025/0185925 A1
(43) Pub. Date: Jun. 12, 2025

(54) SYSTEM AND METHOD OF REMOTE MONITORING OF MULTIPLE VITAL SIGNS

(71) Applicant: BLUE SPARK INNOVATIONS, LLC, Westlake, OH (US)

(72) Inventors: John GANNON, Shaker Heights, OH (US); Frank FEDDRIX, Westlake, OH (US); Matthew REAM, Naperville, IL (US); Sashi KOLLI, Virginia Beach, VA (US); Brad HELFMAN, Solon, OH (US); Jon EAGER, Westlake, OH (US)

(21) Appl. No.: 18/971,654

(22) Filed: Dec. 6, 2024

Related U.S. Application Data
(60) Provisional application No. 63/606,741, filed on Dec. 6, 2023.

(51) Int. Cl. A61B 5/0205; A61B 5/00

(52) U.S. Cl. CPC A61B 5/02055; A61B 5/746

(57) ABSTRACT

A multi-sensor remote patient monitoring platform combines multiple technologies on a patient's own device to dramatically reduce the number of devices a patient needs while also reducing device distribution logistics on health care providers. Continuous temperature monitors can be used to collect frequent temperature data over large periods of time. Additionally, a rPPG device can obtain at least one vital sign data by a remote photoplethysmogram (rPPG) comprising a plethysmogram that is optically obtained via a camera by detection of blood volume changes in the microvascular bed of tissue of the patient. The temperature data and vital sign data can be input into a remote server-side computer system that can analyze the received data for indication of a fever, and provide an alert to the patient or a clinician when the result of the analysis indicates the fever. Such alerts can be clinically beneficial for disease states in which fever can be fatal, particularly for those fevers having fast onset.

### CROSS-REFERENCE TO RELATED APPLICATIONS

This application claims the benefit of U.S. Provisional Application No. 63/606,741, filed Dec. 6, 2023, the entire disclosure of which is hereby incorporated herein by reference.

### BACKGROUND

In recent years there has been a growing interest in active medical technologies that can leverage the increasing power of portable computers, smartphones, and tablets. One such example includes a Body Temperature Logging patch (“patch”) that can be worn on the body to track and collect in memory the temperature of the patient's body over time. Conventional body temperature devices today take a measure of the body temperature at only a single point in time. In contrast, an automated patch device can be worn over a lengthy period of time, such as a 24, 48, or 72 hour period (although longer or shorter time periods are contemplated).

Although temperature is one very important vital sign and indicator of health, there is an increasing need to capture additional vital signs of a patient in an automated or semi-automated manner through one or more additional sensor(s). For example, other vital signs could include any or all of the patient's pulse or heart rate, heart rate variability, blood pressure, blood-oxygen levels (i.e., SpO2), respiratory rate, EKG, ambient temperature, ambient humidity, ambient pressure, ambient light, sound, and/or radiation levels, patient bodily functions, time, patient movement (e.g., via an accelerometer), and/or multiple temperatures of the patient at the same or different locations, etc.

Recent cancer treatments have included various immunotherapies. Among these, chimeric antigen receptor T-cell (CAR-T) therapy involves engineering autologous T-cells harvested from a patient to express the chimeric antigen receptor (CAR) gene. The CAR gene elicits growth of a receptor on the T-cell that binds to special proteins on the surface of a patient's cancer cells. The harvested and then engineered cells are grown in vitro and then infused back into the patient. Once in the patient, the cells further continue to grow logarithmically and attack the patient's cancer cells, which are targeted by the CAR receptor.

CAR-T therapy has proven to be particularly successful in treating liquid tumors (e.g., hematological cancers such as leukemia and lymphomas). Similar T-cell immunotherapies include T-cell receptor (TCR) therapy where the T-cell is genetically engineered to produce a TCR protein that targets antigens inside of the cancer cells; and tumor-infiltrating lymphocyte (TIL) therapy where the harvested T-cells are from a solid tumor biopsy and thus already recognize the cancer cells.

In these therapies, however, the elevated number of T-cells causes an increase in cytokine levels in the patient. Such a cytokine increase can result in cytokine release syndrome (CRS), which is a common toxicity in patients receiving those therapies. CRS is characterized initially by a fever, which can escalate and progress quickly to life-threatening vasodilatory shock, capillary leak, hypoxia, and end-organ dysfunction.

Therefore, earlier detection of CRS may allow for more timely interventions, more effective management of patient symptoms, and the prevention of clinical deterioration, thus enhancing patient safety while undergoing these immunotherapies. Furthermore, by minimizing the risk of CRS-related adverse events, more patients may be able to qualify for life-saving CAR-T and BiTE (bispecific T-cell engagers) cell therapies, potentially on an outpatient basis, or in settings with less access to acute care resources such as in community practices. One of the key indicators of CRS is fever, but patients may also present with hypotension and tachycardia or other indicators which can be detected by various other vital signs of the patient.

### BRIEF SUMMARY

The following presents a simplified summary of example embodiments of the invention. This summary is not intended to identify critical elements of the invention or to delineate the scope of the invention. The sole purpose of the summary is to present some example embodiments in simplified form as a prelude to the more detailed description that is presented later.

In accordance with one aspect, a method comprises the step of receiving temperature data from a continuous temperature monitor worn by a patient, the continuous temperature monitor comprising a flexible patch configured to be attached to the patient's skin, the flexible patch comprising a temperature sensor that is configured to automatically collect at least one temperature of the patient every five minutes during a collection time period. The method further comprises the steps of receiving at least one vital sign data from a rPPG device configured to conduct a remote photoplethysmogram (rPPG) comprising a plethysmogram that is optically obtained by detection of blood volume changes in the microvascular bed of tissue of the patient via a non-contact and non-invasive transdermal optical imaging using a camera to measure and analyze variations in the light reflected off the patient's skin. The method further comprises the step of inputting the received temperature data and the at least one vital sign data to a remote server-side computer system comprising at least a processor and database/memory. The remote server-side computer system is configured to analyze the received temperature data and the at least one vital sign data for indication of a fever during the collection time period, and transmit a notification of a result of the analysis to a personal device of the patient and/or to a clinician. The notification comprises an alert when the result of the analysis indicates the fever.

In accordance with another aspect, a method comprises the step of receiving temperature data from a continuous temperature monitor worn by a patient, the continuous temperature monitor comprising a flexible patch configured to be attached to the patient's skin, the flexible patch comprising a temperature sensor that is configured to automatically collect at least one temperature of the patient every five minutes during a collection time period. The method further comprises the steps of receiving at least one vital sign data from a PPG device configured to conduct a photoplethysmogram (PPG) comprising a plethysmogram that is optically obtained by detection of blood volume changes in the microvascular bed of tissue of the patient, and inputting the received temperature data and the at least one vital sign data to a remote server-side computer system comprising at least a processor and database/memory. The remote server-side computer system is configured to analyze the received temperature data and the at least one vital sign data for indication of a fever during the collection time period, and transmit a notification of a result of the analysis to a personal device of the patient and/or to a clinician. The notification comprises an alert when the result of the analysis indicates the fever.

It is to be understood that both the foregoing general description and the following detailed description present example and explanatory embodiments. The accompanying drawings are included to provide a further understanding of the described embodiments and are incorporated into and constitute a part of this specification. The drawings illustrate various example embodiments of the invention.

### BRIEF DESCRIPTION OF THE DRAWINGS

The foregoing and other aspects of the present invention will become apparent to those skilled in the art to which the present invention relates upon reading the following description with reference to the accompanying drawings, in which:

FIG. 1 illustrates an example system for temperature and vital sign monitoring using a rPPG scan.

FIG. 2 illustrates an example user interface for a dedicated software application (“App”) upon a device screen.

FIGS. 3A-3B illustrate additional examples of the App user interface.

FIG. 4 illustrates an example user interface for performing an rPPG scan.

FIG. 5 illustrates an example clinician dashboard that displays temperature and vital sign data from one or more patients.

FIGS. 6A-6C illustrate additional examples of the clinician dashboard.

### DETAILED DESCRIPTION OF EXAMPLE EMBODIMENTS

Example embodiments are described and illustrated in the drawings. These illustrated examples are not intended to be a limitation on the present invention. For example, one or more aspects can be utilized in other embodiments and even other types of devices. Moreover, certain terminology is used herein for convenience only and is not to be taken as a limitation. Still further, in the drawings, the same reference numerals are employed for designating the same elements. It is to be appreciated that various embodiments are disclosed herein, and that any feature(s) of a particular embodiment may be used interchangeably in any other embodiment. That is, the embodiments are not intended to be mutually exclusive and the features of each embodiment may be utilized in various combinations with some or all of the features of another embodiment.

The instant application provides patient temperature data combined with various other vital sign(s) to enable clinicians and other healthcare professionals to more quickly and effectively diagnose various diseases. For simplicity, the instant application will be described within the example of CRS, although with the understanding that the methods and structure herein are similarly applicable to various other diseases and medical use cases. CRS is treatable when in its initial stages but can become fatal particularly when progressed to Grade 4. CRS is initially characterized by a fever. Therefore, early detection of fever in patients susceptible to CRS can help identify CRS onset so that it may be timely treated. Conventionally, the current standard of care for temperature monitoring is to record a patient's temperature every four hours, or approximately six times per day (i.e., per 24-hour period). However, such a relatively low-frequency temperature check is likely to miss early indications of CRS onset.

Monitoring of a patient's temperature can be done via continuous temperature monitoring. Such continuous temperature monitoring may be realized, for example, with the body temperature logging patch described in any of U.S. Pat. Nos. 11,819,313; 10,849,501; 9,782,082; and/or 9,693,689, all of which are incorporated herein by reference and which are commercially available under the TempTraq® brand. The body temperature logging patch can be worn on the body of a patient, such as on the torso or adjacent to the underarm location (i.e., the armpit or axillary region). It could also be worn at other locations, such as on the forehead, other torso locations, arm, leg, foot, chest, or other on-body location.

The use of continuous temperature monitoring provides much higher frequency collection of patient temperatures. In several non-limiting examples, the above-described body temperature logging patch can record a patient's temperature at a frequency of once every ten seconds, or approximately 8,640 times per day (i.e., per 24-hour period). Various other frequency rates are considered, such as once every thirty seconds (i.e., 2,880 times per day), once every minute (i.e., 1,440 times per day), or once every five minutes (i.e., 288 times per day). Preferably, the frequency rate of temperature recording is a value every 60 seconds or less. Other frequencies of temperature data collection are also contemplated. As can be readily seen, automated and continuous temperature monitoring provides a significant increase in the amount of data available for analysis of the patient's health. Hereinafter, “continuous” temperature monitoring refers to automated collection of temperature data at intervals, preferably regular intervals but irregular intervals are also contemplated. Clinical trials utilizing continuous temperature monitoring have demonstrated fever detection up to 18.5 hours earlier fever detection compared to standard of care in patients with infection and up to 4.4 hours earlier identification of fever in patients at risk for CRS in a limited cohort of patients receiving cell therapies.

Additional vital signs of a patient can be simultaneously captured, together with temperature, in an automated or semi-automated manner through one or more additional sensor(s). Although it is possible to use multiple sensors, there is a desire to utilize as few a number of devices as possible to simplify and reduce the burden upon the patient. It is to be appreciated that the techniques described herein can be performed within a healthcare center (i.e., a hospital or other medical center) or in an outpatient scenario where the patient will be monitored in an environment outside of a healthcare center (i.e., at home). In the outpatient scenario, there are additional technical challenges because the patient is more mobile and must obtain the various vital sign readings without the physical presence of a healthcare professional.

In one embodiment, one or more additional sensor(s) can be combined with or integrated into the body temperature logging patch. For example, the additional sensor(s) could outwardly sense the patient and capture signals from beneath the patient's arm (with arm down), where the temperature sensor is oriented towards the armpit. However, it can be challenging to physically integrate these different sensors because an underarm location typically isn't an optimal place to obtain other vital signs, and an alternate exposed area of the body (e.g., the arm) isn't an optimal place to obtain consistent temperature information. Thus, in another embodiment, the one or more additional sensor(s) could be implemented upon an entirely different device that separately monitors the patient, but which can transmit the captured vital sign data to a common computing device. For example, the separate device could be a patch or a wraparound device (like an arm band).

In one embodiment, the additional vital signs of the patient could be captured by a photoplethysmogram (PPG). This is an optically obtained plethysmogram that can be used to detect blood volume changes in the microvascular bed of tissue. In one example, a PPG can be obtained by using a pulse oximeter which illuminates the skin using infrared or red light, and measures changes in light absorption using a photodetector for specific wavelengths. The PPG technique can be used to obtain at least the patient's pulse or heart rate, heart rate variability, blood pressure, blood-oxygen levels (i.e., SpO2), and/or respiratory rate. However, the use of an on-body PPG commonly requires some form of contact with the human skin, such as the ear, finger, or arm. An on-body PPG can be performed using a patch or a wraparound device (like an arm band) that is physically worn upon the patient's body.

In a preferred embodiment, the additional vital signs of the patient could be captured by a remote photoplethysmogram (rPPG), which is a non-contact and non-invasive method of transdermal optical imaging that uses a camera to measure the patient's vital signs by analyzing variations in the light reflected off their skin. The rPPG technique can be used to similarly obtain at least the patient's pulse or heart rate, heart rate variability, blood pressure, blood-oxygen levels (i.e., SpO2), and/or respiratory rate. Importantly, the rPPG technique is a non-contact method that does not require any contact with the patient's body and therefore avoids the need for a device to be worn upon the patient's body. In one advantageous example, the camera can be the front-facing camera of a smartphone or tablet. In rPPG, the camera is used to capture the patient's blood flow patterns, which are then translated into vital sign measurements, health indicators, and risk profiles. In one example, the rPPG can be used to measure changes in facial blood flow. Additionally, because rPPG uses a camera, the images captured by that camera can also be used for various other applications, including emotion recognition and assessing a patient's stress levels.

The basis of rPPG is the fact that blood absorbs light and becomes more or less translucent depending on the blood volume. When your heart beats, your blood vessels expand and contract, which affects the amount of light absorbed and reflected by the skin. The light surrounding the patient interacts with blood vessels in the deepest skin layers, and different red/green/blue (RGB) wavelengths have different penetration depths. For example, blue light can penetrate the epidermis and derma, green light can penetrate the upper fat layer, red light can penetrate to the deeper fat layer, and near infrared can penetrate to the muscle layer. As noted above, the rPPG technique uses an electronic camera, for example a camera integrated into a smartphone, tablet, webcam, or laptop/desktop computer, to capture these RGB wavelengths. PPG and rPPG both measure the volumetric change by reading the light transmission or reflection captured by diodes on the skin or by analyzing RGB signal changes, for example, in 1800 frames of video (60 sec×30 fps), respectively.

There are several factors that can influence an rPPG scan. Typically, the rPPG system examines the vasculature below four different landmarks on the patient's face: above the eyebrows (left and right) and the cheeks (L/R). These points are typically unaffected by facial hair, except for exceptionally thick facial hair or bangs that cover both the forehead down past the cheeks can interfere with the reading if not pulled back from the face. These four points also create redundancies that allow accurate calculations around scarring using the unaffected point (though accuracy may again be affected by extensive scan tissue covering forehead and cheeks). The following are key factors that can impact the accuracy of a scan. Light Source: light source, such as LED provide high quality lighting onto the person's skin. This light can be in the visible or near-infrared spectrum. Camera or Sensor: A camera or webcam is used to capture the light that is reflected back from the person's skin. This sensor records variations in the intensity of reflected light over time. Signal Processing: The collected data is then subjected to signal processing algorithms. These algorithms analyze the variations in light intensity to extract physiological information. The algorithm looks for periodic changes in light intensity that correspond to the person's heart rate.

Temperature is the only vital sign that rPPG technology is not capable of obtaining, making the pairing of continuous temperature monitoring and rPPG innovative and novel. Thus, the instant application provides a multi-sensor remote patient monitoring platform, that integrates a core body temperature wearable with contactless vital sign measurement sensors. Combining the two technologies on a patient's own device dramatically reduces the number of devices a patient needs while also reducing device distribution logistics on health care providers.

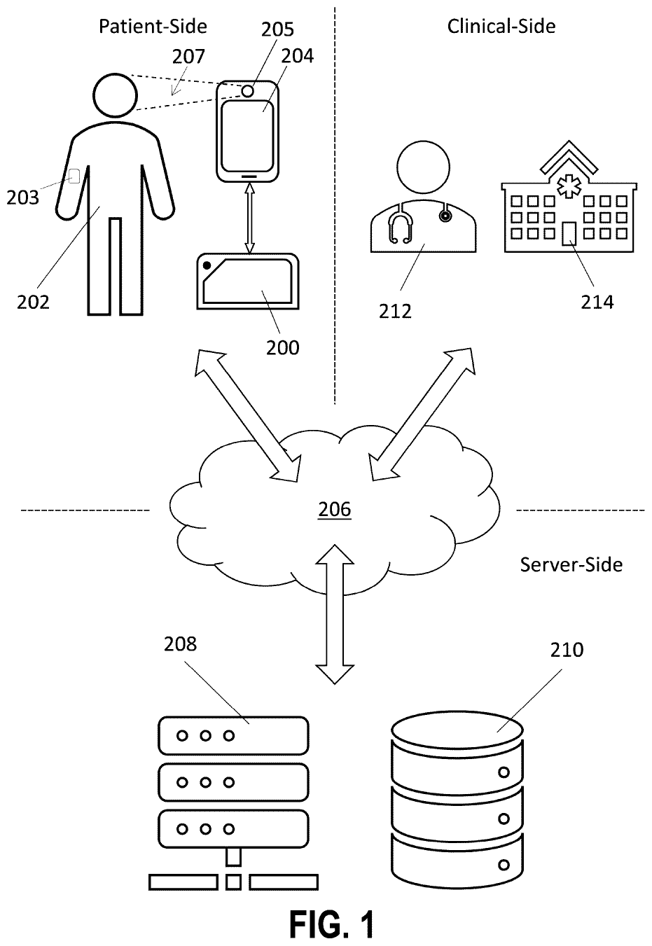

Turning to FIG. 1, temperature data is collected at a patient-side from a continuous temperature monitor 200 on a patient 202. The patient 202 may monitor the collected temperature data in real-time, along with previously detected temperature data, trends, and the like, on a personal device 204 (e.g., smart phone, tablet, computer, or the like). The personal device 204 may communicate with the monitor 200 via any short range wireless communication protocols including, for example, Bluetooth, near field communication, and the like.

Additionally, during a same collection time period as the temperature data is collected, at least one vital sign data is collected at the patient-side from a rPPG device that is configured to conduct a photoplethysmogram (PPG), preferably a remote photoplethysmogram (rPPG). The vital sign data can be at least one of the patient's pulse or heart rate, heart rate variability, blood pressure, blood-oxygen levels, and/or respiratory rate. Optionally, other vital signs may also be collected from other wired or wireless sensors, such as the patient's weight, blood pressure from a physical cuff device, EKG readings, etc. Preferably, the photoplethysmogram is capable of obtaining multiple vital signs. In one example, as will be described in more detail, the photoplethysmogram (PPG) can be a remote photoplethysmogram (rPPG) comprising non-contact and non-invasive transdermal optical imaging 207 using a camera 205 to measure and analyze variations in the light reflected off the patient's skin. The camera is part of the rPPG device comprising at least one of a smartphone, tablet, or other computer device, and preferably, is the personal device 204 (e.g., smart phone, tablet, computer, or the like) of the patient. In this case, the camera 205 can be part of the personal device 204, such as the front-facing camera of a smartphone, tablet, or laptop computer. The camera could also be a rear-facing camera of the personal device, or could even be an external camera that is connected to the personal device via a wired or wireless connection. The rPPG device (e.g., the patient's personal device 204) can comprise a processor and non-transient memory that is configured to execute a computer program application to operate the camera 205 and obtain the at least one vital sign data by conducting the remote photoplethysmogram using the camera 205.

The collected temperature data and at least one vital sign data is then transmitted over a network (e.g., the Internet) 206 and input into a server-side computer system 208. As such, the server-side computer system 208 provides a platform that enables multiple inputs of data from the patient to be collected, correlate, and interpreted together. The computer system 208 comprises a processor, database/memory, and the like. The computer system 208 is configured to perform analysis on the received temperature data and the at least one vital sign data. The analysis can be done by various programmed algorithms, comparison to threshold values, etc. In a further example, the server-side computer system 208 can be configured at least in part as a machine learning system, such as a Multi-step Multi-layer Perceptron (MLP) network. An MLP is a type of feedforward neural network, which is robust to noisy input data and capable of learning linear and nonlinear relationships in training data. Further, because MLPs can provide multiple outputs, the MLP can produce a forecast for multiple future time points. However, because MLPs have a fixed input and output, the MLP has a pre-determined temporal dependence. In other words, the time periods of input and output temperature data (the number of input and output points) is pre-determined.

In still other examples, the machine learning system may include Convolutional Neural Networks (CNNs) including Univariate Multi-step CNNs and Temporal Convolutional Networks (TCNs), Long-Short-Term Memory (LTSM) models (which can learn the temporal dependence), and/or Sequence to Sequence (S2S) models. Such systems may be trained with training data similar to that with respect to an MLPs.

Continuing with the server-side computer system 208, the database/memory is configured to store temperature data received from the monitor 200 and the at least one vital sign data received from the rPPG device, and further to process the collected/stored data on the server-side computer system 208. In one example, the data can be processed with the machine learning system. In other embodiments, the temperature data and at least one vital sign data may be stored at a remote database 210 from the machine learning system/computer system 208. In other embodiments, the computer system 208 including any data processing and/or analysis may additionally or alternatively be implemented in part or in whole locally at the patient-side, for example, on the patient's personal device 204.

In one form, after optionally being subject to optional pre-processing, the received temperature data may be compared to a threshold temperature to determine whether the patient 202 is currently experiencing (or had previously experienced) a fever. In a similar form, after optionally being subject to pre-processing, the received at least one vital sign data may also be compared against relevant threshold value(s) to determine whether the patient 202 is currently experiencing (or had previously experienced) a fever, or possibly another anomalous medical condition. In addition or alternatively, the received temperature data may be compared to a rate of change (e.g., increase or decrease) of temperature exceeding a predetermined threshold, a period of time of the temperature data is above a predetermined threshold, or the like. A similar analysis of the rate of change (e.g., increase or decrease) can be performed for the at least one vital sign data, i.e., the rate of change exceeding a predetermined threshold or a period of time of the temperature data is above a predetermined threshold.

In the event the patient 202 has a fever or other medical condition, the computer system 208 may transmit an alert to the patient's personal device 204 at the patient-side and/or a doctor 212, hospital 214, or like clinician at a clinical-side, via network 206. These alerts may further include instructions to the patient to, for example, seek medical attention. The output may also include the received temperatures and/or a temperature profile for review and analysis by the doctor 212 at the clinical-side.

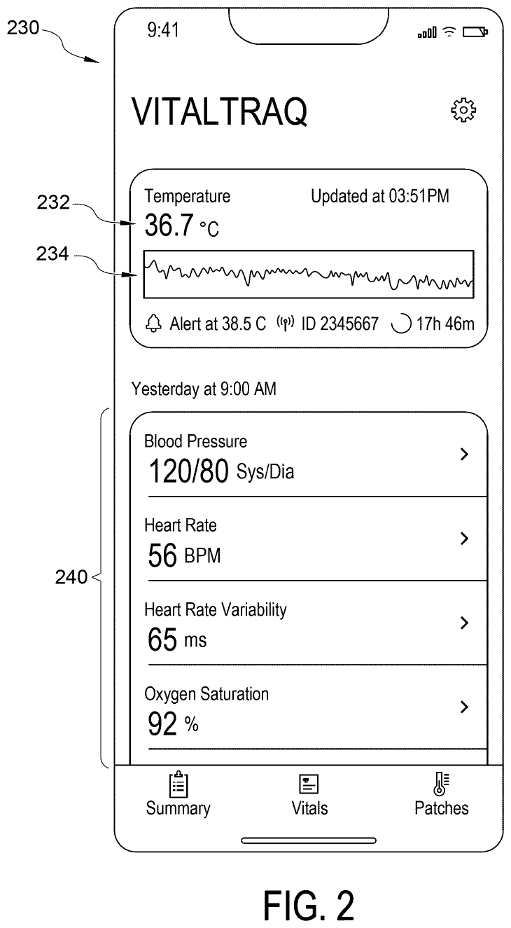

Turning now to FIG. 2, the rPPG facial scan can be performed in various ways. Although one method will be described for use with a handheld smartphone as the rPPG device (e.g., the personal device 204 of the patient 202), it is to be understood that a similar method can be used with a tablet, computer, or other electronic device. The rPPG device (e.g., the personal device 204 of the patient 202) comprises a processor and non-transient memory that is configured to execute a computer program application, i.e., a dedicated software application (“App”). First, the user will unlock the smartphone 204 and launch the App 230 that will be displayed upon the screen of the smartphone. Preferably, the App will already have the user's personal account loaded, but if not, then an option will be presented so that the user can log into a dedicated account. If the user has previously used the App to perform previous scans, the App may inform the user when the next scheduled facial scan should occur, or if the scan is overdue. Additionally, the App can also provide access to the patient's previous scan results.

The App will communicate with the body temperature logging patch 200, for example, as described in U.S. Pat. Nos. 10,849,501 or 9,782,082 via any short range wireless communication protocols including, for example, Bluetooth, near field communication, and the like. That is, the App will capture previously logged or even instantaneous temperature readings captured by the body temperature logging patch being worn by the patient. These current temperature readings 232 will be displayed upon the smartphone screen, optionally with previous data 234, trends, and the like.

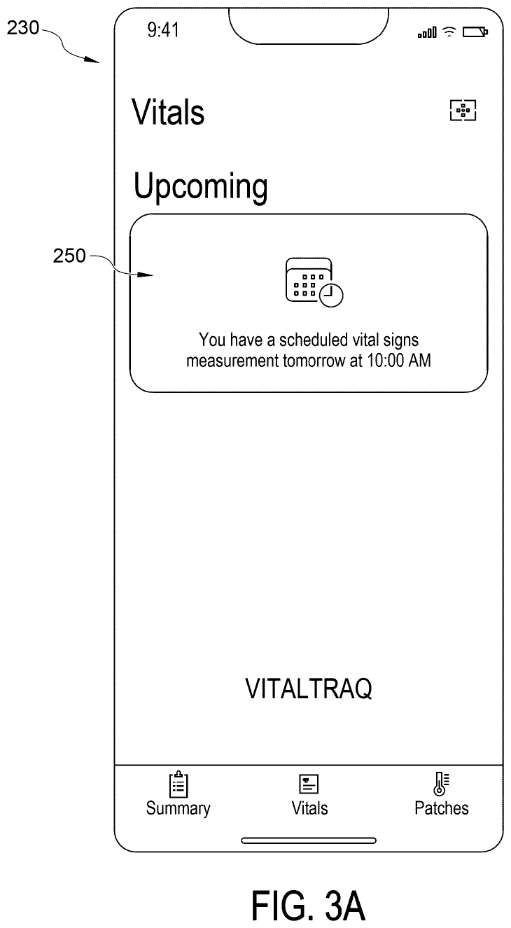

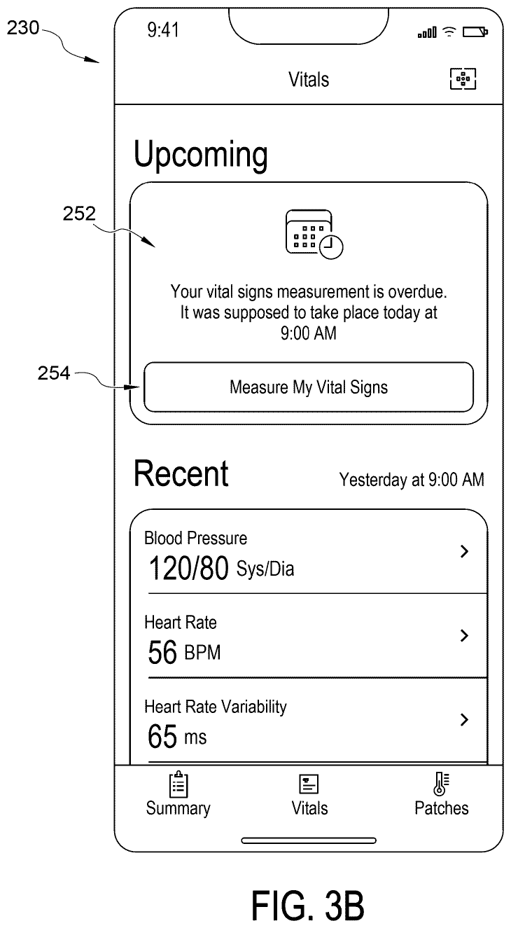

Turning to FIGS. 3A-3B, in one example, the App can further comprise a clock and/or clock program that can be configured to provide the patient at least one of a reminder 250 to conduct the photoplethysmogram at predetermined time intervals, or an alert 252 that a photoplethysmogram has been missed. For example, the clock program can provide the patient a reminder 250 to conduct the rPPG scan every four hours, and may further include a countdown timer or other visual display so that the patient can know how long until their next scan should occur. The clock program can also provide the patient with an alert 252 (visual or audible) that indicates that a previously-scheduled photoplethysmogram has been missed so that the user can take appropriate action to conduct the scan.

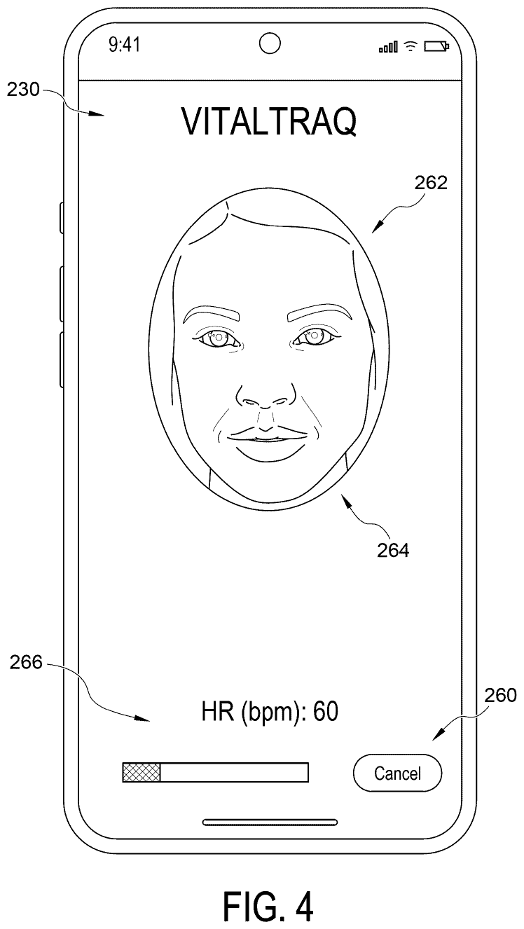

As shown in FIG. 3B, App will provide a button 254 on the screen to activate a Vital Signs rPPG facial scan, which will be performed by the front-facing camera 205 of the smartphone 204. Turning to FIG. 4, the App is further programmed to operate the camera 205 and will prompt the user to hold the smartphone at an arm's length in front of their face and in full and unobstructed view 207 of the camera 205, similar to taking a “selfie” picture, and to hold the smartphone steady for about 60 seconds. Upon starting the facial scan the smartphone App will begin the rPPG facial scan using the front-facing camera. The user interface can provide various controls 260, such as a “start measurement” button or a “cancel” button on the screen. Preferably, the screen of the smartphone will display a live video 262 feed of the patient's face that is overlaid with alignment indicia 264 to help ensure that the patient has properly oriented the camera. In one example, the alignment indicia 264 can present an oval shape to assist the patient to maintain their entire face is located within a predetermined target zone prior to and during the rPPG scan. More preferably, the App may overlay helpful prompts and information 266, such as one or more vital signs being detected in real time. Other prompts and information may help to indicate the current progress of the scan, whether the quality of the scan appears to be good or bad, and/or may further make suggestions to the patient of ways to increase the quality of the scan (for example, to hold still, re-orient the camera, and/or use better lighting). Still further, alerts related to any errors in receiving temperature readings or facial scan data, or the occurrence of abnormal data (e.g., low temperature readings/facial scan errors), can be displayed upon the smartphone screen. Optionally, suggestions can be given to the patient of ways to increase the quality of the temperature readings, such as to re-position the temperature logging patch to a better location. Accordingly, the patient may be made aware of operation, connection, or like problems with the temperature patch or facial scan, and how those problems may be corrected.

Typically, the App may only display the final results upon the screen once the facial scan has completed. Optionally, the App may also display any instantaneous (i.e., real-time or as close to real time as practically possible) data upon the screen, for example, from one or more locations on the patient's face. The locations may be indicated by indicia so that the patient understands which portions of their face are being actively analyzed. The displayed data may be in numerical form and/or graph form. Additionally, the App will collect instant temperature data from the body temperature logging patch to correlate the rPPG data from the facial scan together with the temperature data collected during that same scan. The App may request a higher frequency of temperature data from the body temperature logging patch, or may simply take one or several readings immediately before and/or after the rPPG facial scan. Upon completion of the rPPG scan, the App may display the instantaneous and, upon request, historical values of each vital sign that is detected so that the patient can compare the instant result to previous scans.

In one example, a patient will use the App to perform a 30 second facial scan 4-5 times a day during the monitoring period, e.g., at predetermined time intervals, which preferably are consistent with the collection time period where temperature data is collected from the continuous temperature monitor The facial scan will assess the patient's non-temperature vital signs, such as blood pressure, heart rate, heart rate variation, and respiratory rate. The patient will scan their face approximately every 4 hours for a 16 hour period, beginning when they wake up. Patients will be instructed to perform the scan at least 4 times per day within the 16 hour window, allowing a variability of 4-5 scans per day so as not to inhibit a full night of sleep.

Optionally, in the case of a rPPG technique, incentives can be used to encourage outpatients to conduct a facial scan in order to obtain the vital sign readings. In one example, the App or an associated computer application (i.e., a smartphone app, computer program, etc.) can include gamification methods to encourage the patient. Various gamification methods can include notifications when it is time to take a facial scan, and/or animations, newsfeeds, trivia, face filters, videos, points for successful scan, etc. while the facial scan is occurring (i.e., when it is taking rPPG images). Alternatively, it may be preferable to have the smart phone app conduct automated scans whenever the phone operated to thereby avoid the need to rely upon the patient. In one example, such automated scans could occur on a predetermined time schedule. In another example, the automated scan could start automatically when the patient unlocks their phone for the first time after the next scheduled facial scan is due to occur. In other examples, the smartphone app can provide subtle feedback to indicate whether the facial scan is working or not, and potentially what actions or adjustments the patient could take to improve the quality of the facial scan. Lastly, once the facial scan is done, the results can be shared with the patient upon the device screen (i.e., the smartphone or computer screen). Optionally, if the patient has a medical caregiver, then reminders and/or alerts can be sent to the caregiver to encourage additional compliance with the scan schedule.

In another example where the rPPG technique is performed in a healthcare setting, a dedicated recording station can be provided. This can provide an advantage whereby one or several recording stations can be used for any number of patients that visit that healthcare provider, without requiring a separate smartphone/tablet/computer for the patient. That is, the recording station can be a stationary or movable pedestal that contains a camera, video screen, and computing device. Preferably the station can further include input peripherals such as a keyboard and/or mouse. A patient can walk up to the device, identify themselves (for example via the input peripherals) and perform a rPPG scan as described herein. The results can be displayed upon the video screen and the data automatically captured and provided to the proper healthcare professional via a computer network. When the scan is finished, the recording station then be used by another patient. Because the rPPG technique is a non-contact method, this provides an advantage whereby there is a reduced need to clean and/or sanitize portions of the recording station.

Although the above method is described for use with a smartphone and rPPG technique, it is to be understood that a similar methodology can be used for a skin-contact PPG sensor. For example, the described method may further comprise an on-body sensor that is physically worn upon the patient's body to conduct an auxiliary photoplethysmogram (PPG) scan. As described here, such a skin-contact PPG sensor (e.g., on-body sensor 203) could be attached to the patient via an armband or other device and connected to the smartphone App via any short range communication including, for example, Bluetooth, near field communication, and the like. The App can conduct a vital signs scan in a similar manner to collect and correlate the temperature data and vital signs data together. Additionally, it is to be appreciated that other patient vital sign data could be collected by the rPPG device 204 from one or more other medical devices that are wired or wirelessly-connected to the rPPG device 204. Some examples may include a weight sensor to capture the patient's weight, a blood-pressure cuff to physically capture the patient's blood pressure in a more traditional technique, an EKG or ECG sensor, respiratory rate, ambient temperature, ambient humidity, ambient pressure, ambient light, sound, and/or radiation levels, patient bodily functions, time, patient movement or fall detection sensor (e.g., via an accelerometer), and/or multiple temperatures of the patient at the same or different locations, etc.

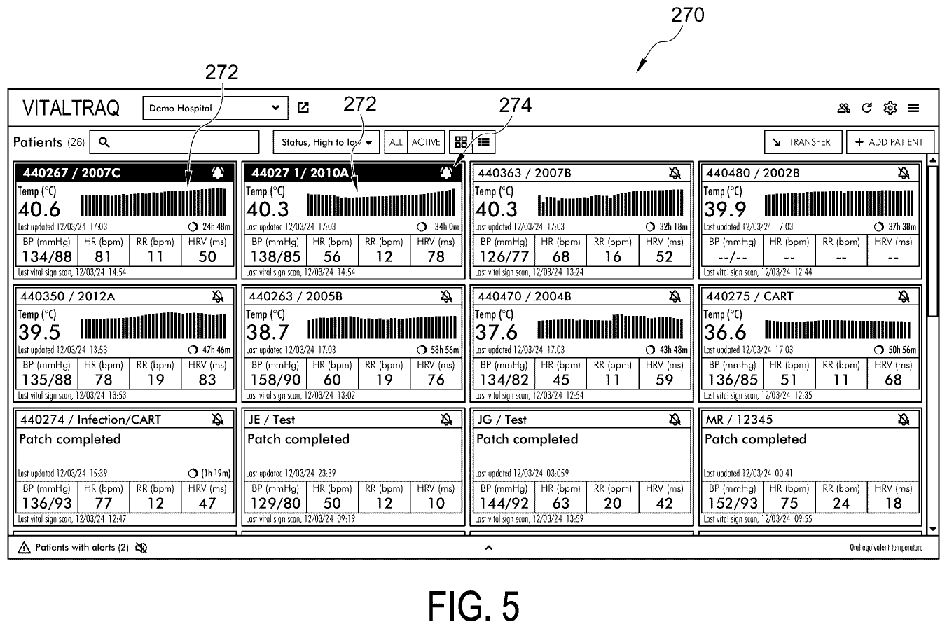

Turning to FIG. 5, regardless of the rPPG or PPG vital signs scan, the resulting data can be transmitted over a network (e.g., a local network or the Internet) to a computer system for additional processing and/or display. For example, the data for display can be provided by the remote server-side computer system to a clinician's screen at a hospital or other location. In one embodiment, a healthcare provider can be provided with a visual clinician dashboard 270 that displays the temperature and vital sign data from one or preferably multiple patients. Often the clinician dashboard 270 will be displayed upon a computer screen or the like, such as on a stationary or mobile computer device. Preferably, the temperature data and at least one vital sign data are displayed in substantially real time with the receipt of such data by the remote server-side computer system. In one example, the dashboard can be provided in a grid format that displays the most recent temperature data and/or vital sign data for a plurality of patients in the form of numerical values and/or a time-series graph 272, and/or a narrative indicating the patient's current or predicted future condition. The data can be organized variously. In the illustrated example, the most recent temperature reading can be displayed next to a historical graph of the temperature readings over time. Underneath the temperature information, one or more detected vital signs can be provided. For example, the patient's most recent blood pressure, heart rate, heart rate variability, and respiratory rate. Other vital sign data can also be shown.

The data can be color-coded to indicate normal readings (e.g., green) or readings which require additional review (e.g., yellow) or even urgent review (e.g., red) by a healthcare professional. Optionally, additional alerts 274 can be displayed for patients that appear to need immediate review. This easily permits the healthcare professional to understand and triage the status of multiple patients at a glance. Although the dashboard shows visual data, the alerts can be combined with audible or vibratory alarms as well. Lastly, each patient visualization can include various time indicators, such as an indication of the last time the data was updated, how long the patient has been monitored, how long the current temperature logging patch has been active, etc.

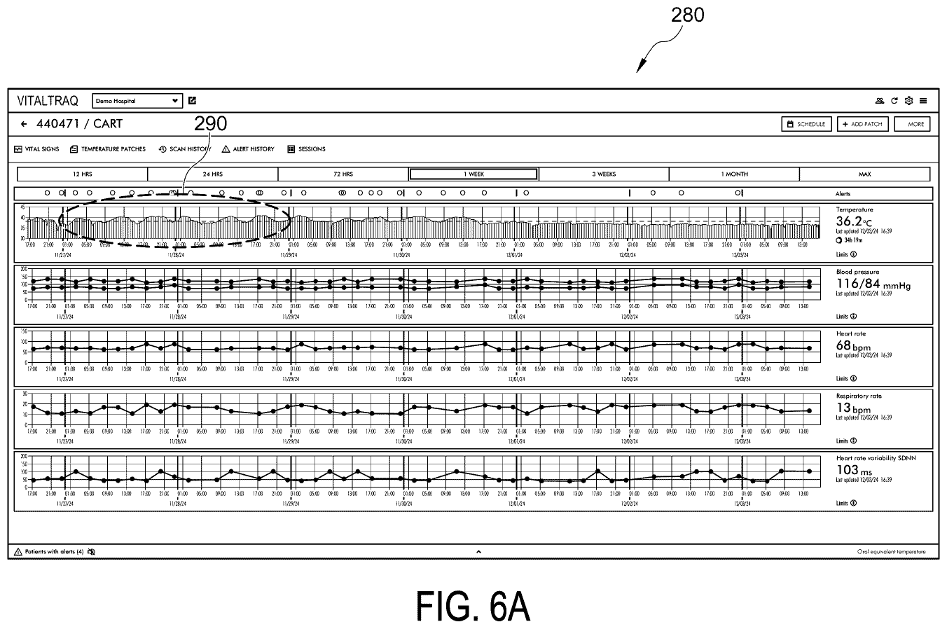

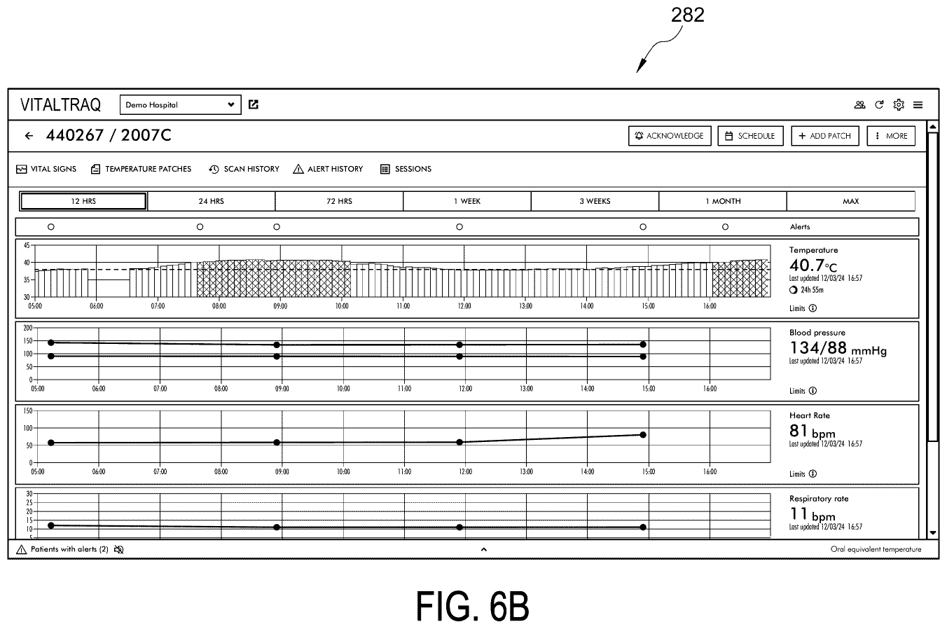

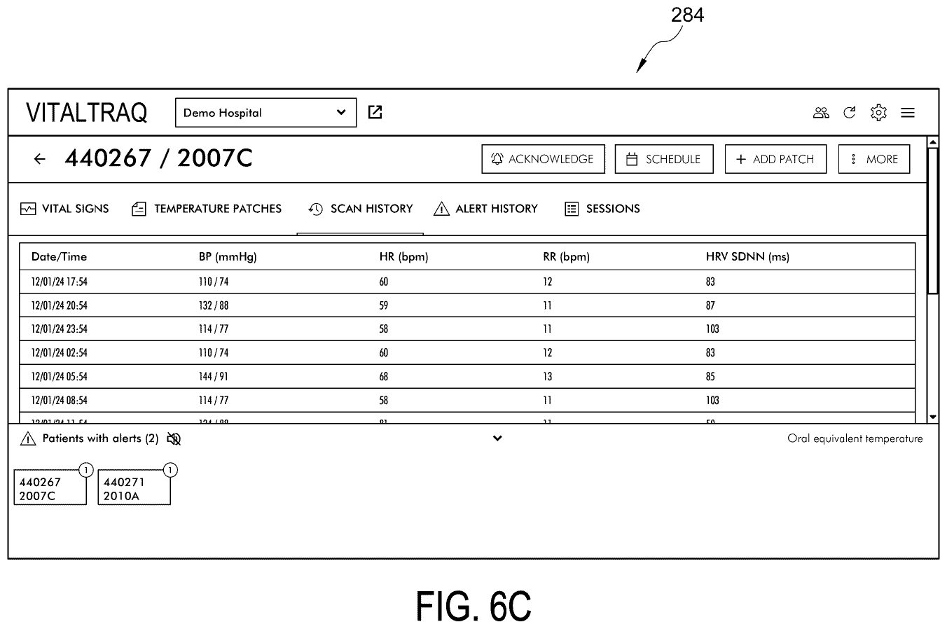

Turning to FIG. 6A, upon request, the data for each individual patient in the grid can be expanded to reveal more a detailed view 280 of the temperature data, vital signs and/or historical data of only that one patient. This enables the clinician to have a more detailed and granular view of the patient's medical history as captured by the temperature patch and rPPG scans. In the example shown in FIG. 6A, the detailed view can show the patient data over various time periods, for example, partial days, entire weeks, or a month (or more). The ability to view the patient data over different time periods enables the clinician to observe trends in the data. Preferably, as shown in FIG. 6A, the various data (temperature and each vital sign) will be time-correlated and arranged in a series of rows so that the clinician can observe patient's condition via the interaction of various data. For example, as shown in FIG. 6A, the temperature data can be shown over time in a first row, and below that over the same time period the patients various vital sign data can be shown, such as blood pressure, heart rate, respiratory rate, heard rate variability, etc.

In one example, a clinician could review the temperature data shown in FIG. 6A while evaluating a patient for cytokine release syndrome (CRS), which is characterized initially by a fever above 38.5° C. that can escalate and progress quickly to life-threatening conditions. Often, as shown in area 290, an onset of CRS is also characterized by a series of rapid temperature increase (i.e., fever onset) and then rapid temperature reduction, which often happens as a number of cycles. Additionally, because the various vital signs are time-correlated with the temperature data and displayed on the same detailed view 280, the clinician can observe many other aspects of the patient's medical condition at the time of the increased temperature. For example, it can be seen in FIG. 6A that many of the vital signs also increased at similar times during which the patient's temperature increased, and likewise decreased with temperature decrease over the various cycles as a pattern. The detailed view 280 that combines the temperature data together with the vital sign data provides a multi-faceted and holistic graphical view of the overall patient's medical state over time that enables a clinician to interpret and make a diagnosis, such as the onset of CRS. It is to be appreciated that the computer system 208 may be configured to analyze multiple data points of the temperature data and/or vital sign data over time to suggest that the patient is experiencing a fever event, taking into consideration past experience (i.e., past temperature and/or vital sign data) of the patient. The computer system 208 may further be trained to recognize the temperature cycles and/or utilize other pattern matching techniques on the temperature and/or vital sign data over time to provide alerts of a patient fever, and/or to make other suggestions to assist the clinician's interpretations and diagnosis.

As shown in FIG. 6B, the dashboard can provide a “zoom in” view 282 of the chart shown in FIG. 6A to provide the ability to view only a restricted amount of time (e.g., 12 hours) if the clinician wishes to evaluate a specific time period. This enables a highly granular and customizable review and evaluation of the patient's temperature data and vital sign data in that fine-tuned time period. The numerical values of each data point at a selected time point can be displayed towards one side of the view 282. In addition or alternatively, as shown in FIG. 6C, the dashboard can further display the patient's temperature data and vital sign data in a numerical chart 284 of datapoints, for example the values from each of a series of previous rPPG scans, which can enable other types of analysis by the clinician. Using the data shown in the various dashboard views, the clinician can form a medical diagnosis.

In the event the patient has a fever or the vital signs indicate another abnormal condition, the computer system may transmit an alert to the patient's personal device at the patient-side and/or a doctor, hospital, or like clinician at the healthcare provider-side. These alerts may further include instructions to the patient to, for example, seek medical attention. The output may also include the received vital signs for review and analysis by the doctor at the healthcare provider. In this manner, the patient and clinicians may be kept aware of the patient's condition in real time as the temperature data and rPPG/PPG data is collected together.

Based on the greatly increased amount of temperature data collection together with the additional vital signs from PPG or rPPG techniques, advanced clinical analysis can be performed for a patient to determine a relationship between temperature, vital signs, and the patient's health state over time. In other words, by correlating the data from several vital signs, profiles can be identified that can indicate the patient's general state of health, or even various health conditions and/or to identify and diagnose particular types of fevers and diseases. In other examples, health data captured over a period of time can indicate trends in the patient's health which may not be otherwise readily apparent and/or which may be used to predict future outcomes. For example, the identification/diagnosis of fevers and diseases by way of correlating temperature data with other vital signs can be based, at least in part, upon data that is accumulated over a particular time period with the above-noted continuous temperature monitors and periodic PPG or rPPG scans. Such data may be processed automatically by computer analysis, or assembled into a graphical format for visual analysis by a clinician. Conveniently, the graphical format can readily be viewed and understood by doctors, nurses, or other hospital staff for confirmation of the automated computer analysis.

Any of the outputs described herein may be provided in as close to real time as practically possible. That is, the computer system 208 may be configured to process any temperature data and/or at least one vital sign data upon receipt, and conduct any subsequent analysis and produce any of the desired outputs upon the determination of the prediction temperature data (e.g., from the machine learning system). For example, as suggested above, temperature data and/or at least one vital sign data may be transmitted to the computer system 208 from the monitor 200 or rPPG device 204 at intervals corresponding to the frequency of temperature collection by the monitor 200 and/or PPG scan by the rPPG device 204. By processing this received temperature data and/or at least one vital sign data upon receipt, the patient 202, doctor/clinician 212, and/or hospital 214 may be notified by the output (e.g., an updated predicted temperature profile or fever alert) at substantially the same frequency. In this manner, the patient and clinicians may be kept aware of the patient's condition in real time as the temperature data is collected. For example, where there are temperature readings or vital sign readings that exceed a predetermined threshold, alerts can be given in real time (or as close to real time as practically possible) so that the medical clinicians can quickly interpret the data and make fast medical decisions.

Although the above description relates primarily to CRS and CAR-T, the scope of the present disclosure is not so limited and may relate to different temperature profiles and risk factors associated with other conditions and diseases. For example, these other conditions and diseases may include sepsis; autoimmune diseases, such as rheumatoid arthritis, Crohn's disease/colitis, lupus, Behcet syndrome, blood clots, deep vein thrombosis, and pulmonary embolisms; neurological disorders such as spinal cord injury and stimulant overdoses; psychological disorders; neoplasms such as lymphoma, leukemia, and hypernephromas; endocrine disorders such as hyperthyroidism and adrenal insufficiency; and transfusion reactions. Therefore, similar continuous temperature monitoring and analysis of the resulting temperature data and corresponding risk factors can also be used for diagnosis of those conditions.

The invention has been described with reference to the example embodiments described above. Modifications and alterations will occur to others upon a reading and understanding of this specification. Examples embodiments incorporating one or more aspects of the invention are intended to include all such modifications and alterations insofar as they come within the scope of the appended claims.

What is claimed is:

1. A method comprising:
receiving temperature data from a continuous temperature monitor worn by a patient, the continuous temperature monitor comprising a flexible patch configured to be attached to the patient's skin, the flexible patch comprising a temperature sensor that is configured to automatically collect at least one temperature of the patient every five minutes during a collection time period;
receiving at least one vital sign data from a rPPG device configured to conduct a remote photoplethysmogram (rPPG) comprising a plethysmogram that is optically obtained by detection of blood volume changes in the microvascular bed of tissue of the patient via a non-contact and non-invasive transdermal optical imaging using a camera to measure and analyze variations in the light reflected off the patient's skin;
inputting the received temperature data and the at least one vital sign data to a remote server-side computer system comprising at least a processor and database/memory,
wherein the remote server-side computer system is configured to:
analyze the received temperature data and the at least one vital sign data for indication of a fever during the collection time period; and
transmit a notification of a result of the analysis to a personal device of the patient and/or to a clinician,
wherein the notification comprises an alert when the result of the analysis indicates the fever.

2. The method of claim 1, wherein the at least one vital sign data comprises at least one of the patient's pulse or heart rate, heart rate variability, blood pressure, blood-oxygen levels, and/or respiratory rate.

3. The method of claim 1, the camera is part of the rPPG device comprising at least one of a smartphone, tablet, or other computer device.

4. The method of claim 3, wherein the camera of the rPPG device is a front-facing camera that is arranged on the same side as a video display screen of the rPPG device that displays a live video feed of the patient's face when the remote photoplethysmogram scan is conducted.

5. The method of claim 4, wherein alignment indicia is overlaid onto the live video feed to assist the patient maintain their face within a predetermined target zone during the remote photoplethysmogram scan.

6. The method of claim 3, wherein the rPPG device is the personal device of the patient.

7. The method of claim 3, wherein the rPPG device comprises a processor and non-transient memory that is configured to execute a computer program application to operate the camera and obtain the at least one vital sign data by conducting the remote photoplethysmogram using the camera.

8. The method of claim 7, wherein the computer program application comprises a clock and is configured to provide the patient at least one of a reminder to conduct the photoplethysmogram at predetermined time intervals, or an alert that a photoplethysmogram has been missed.

9. The method of claim 8, wherein the predetermined time interval comprises the collection time period, and wherein the collection time period is four hours long.

10. The method of claim 1, wherein the method further comprises an on-body sensor that is physically worn upon the patient's body to conduct an auxiliary photoplethysmogram (PPG) scan.

11. The method of claim 1, wherein the notification from the remote server-side computer system is transmitted in substantially real time with the receipt of the temperature data, the at least one vital sign data, and result of the analysis.

12. The method of claim 1, wherein each of the flexible patch and the rPPG device comprise a wireless transmitter, and wherein the remote server-side computer system is remotely connected to the continuous temperature monitor, the rPPG device, and to the clinician via a network.

13. The method of claim 1, wherein the patient is undergoing or has undergone chimeric antigen receptor T-cell (CAR-T) therapy, and the fever is caused by cytokine release syndrome.

14. The method of claim 1, wherein analyzing the temperature data and the at least one vital sign data for indication of a fever comprises comparing the temperature data and the at least one vital sign data to respective predetermined thresholds.

15. The method of claim 1, wherein analyzing the temperature data and the at least one vital sign data for indication of a fever comprises comparing a rate of increase of the temperature data and the at least one vital sign data to respective predetermined thresholds.

16. The method of claim 1, wherein the notification from the remote server-side computer system further comprises an instruction to the patient to seek medical attention when the result of the analysis indicates the fever.

17. The method of claim 1, wherein the remote server-side computer system further provides a visual clinician dashboard that displays the temperature data and at least one vital sign data from one or more patients upon a computer screen.

18. The method of claim 17, wherein the temperature data and the at least one vital sign data are time-correlated on the visual clinician dashboard.

19. The method of claim 17, wherein the remote server-side computer system causes the visual clinician dashboard to display the temperature data and at least one vital sign data in substantially real time.

20. The method of claim 17, wherein the visual clinician dashboard further displays the alert when the result of the analysis indicates the fever.
